(19) United States
(12) Patent Application Publication
(10) Pub. No.: US 2026/0069019 A1
(43) Pub. Date: Mar. 12, 2026

(54) MULTI-FUNCTIONAL SKINCARE APPLICATOR

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Chen Kunjie, Shenzhen (CN)

(73) Assignee: SHENZHEN NUON MEDICAL EQUIPMENT CO., LTD., Shenzhen (CN)

(21) Appl. No.: 19/319,796

(22) Filed: Sep. 5, 2025

(30) Foreign Application Priority Data
Sep. 6, 2024 (CN) 2024221925302

(51) Int. Cl. A45D 34/04; A61N 5/06

(52) U.S. Cl. CPC A45D 34/041; A61N 5/0616; A45D 2200/155; A45D 2200/202; A45D 2200/207; A61N 2005/0626; A61N 2005/0644; A61N 2005/0652

(57) ABSTRACT

The present invention relates to an applicator head for applying liquid from a liquid supply container to a user's skin. The applicator head includes a cover, a base, a main body, and a roller. The base has a connecting sleeve with a connecting hole for communication with the container. The main body is rotatably connected to the base and defines a liquid supply channel extending to a mounting groove. A liquid supply tube delivers liquid to the groove, where the roller is rotatably mounted to apply the liquid. Rotation of the main body relative to the base alternately establishes or blocks fluid communication, thereby preventing leakage when not in use. A sealing gasket and stepped retention surfaces provide reliable sealing and secure assembly. The main body further integrates a circuit board, power supply, and phototherapy lamp with light-emitting elements, enabling combined liquid application and light therapy treatment.

### TECHNICAL FIELD

The present invention relates to the field of skincare and cosmetic devices, and more particularly to an applicator head and applicator device for applying liquids such as creams, serums, or cleansers onto the skin. The invention further relates to devices that integrate controlled liquid dispensing, massage rollers, and optional phototherapy functions within a single, ergonomically designed applicator for improved skin treatment and ease of use.

### BACKGROUND

Skincare and cosmetic products, such as creams, serums, and facial cleansers, are widely used for improving skin condition and appearance. Conventionally, these products are applied manually using fingers, sponges, brushes, or simple applicator heads. While such methods allow direct application, they often lack precision in controlling the amount of liquid delivered, resulting in inconsistent application, product wastage, and potential contamination of both the product and the user's skin.

To overcome these drawbacks, some devices have been developed that incorporate functional elements such as rollers, massage heads, or light therapy modules. These devices aim to improve the absorption of the product and enhance skin treatment effects. However, in most conventional designs, the applicator heads are directly connected to the liquid container without any mechanism to control the flow of liquid during storage or transport. As a result, accidental leakage or spillage is common, causing inconvenience to the user and contamination of surrounding objects.

Further, conventional roller-based applicators often have a limited ergonomic design. Rollers may be small, short, or rigid, which reduces the efficiency of liquid application and can be uncomfortable for prolonged use. The shape and orientation of the applicator body are typically not optimized for handling, which may lead to uneven application of the product across the skin surface.

Existing devices that integrate light or phototherapy functionality generally do so in a fixed arrangement, without consideration for shielding, alignment, or user comfort. Light-emitting elements may not be positioned optimally for effective therapy, and the overall design is often bulky, making portability and convenient storage challenging.

Moreover, many prior art devices do not provide an integrated solution combining controlled liquid dispensing, ergonomic rollers, and phototherapy in a single compact unit. Users are often required to use multiple separate devices to achieve comparable effects, which is inconvenient and inefficient.

Therefore, there exists a need for an improved skincare applicator that addresses the above limitations by providing a controlled, leak-resistant liquid supply system, a long and ergonomically designed roller, optional phototherapy functionality, and a compact, portable design, thereby enhancing user convenience, application efficiency, and overall effectiveness of skincare treatment.

### OBJECTS OF THE INVENTION

An object of the present invention is to provide an applicator head and applicator device that can precisely control the flow of liquid from a supply container to the applicator, thereby preventing accidental leakage and spillage during storage, transport, or non-use.

Another object of the invention is to provide an applicator head with a flattened main body and an elongated roller, designed for ergonomic handling and efficient application, ensuring uniform distribution of the liquid across the skin and enhancing user comfort during use.

A further object of the invention is to provide an applicator device that integrates a phototherapy lamp with shielded light-emitting elements, allowing simultaneous skincare treatment with controlled light therapy while maintaining safety and effectiveness.

Yet another object of the invention is to provide a modular and compact design in which the main body, roller, mounting bracket, circuit board, battery, and sealing mechanisms are efficiently arranged to allow easy assembly, maintenance, and portability.

Still another object of the invention is to provide an applicator device that combines liquid dispensing, massage, and phototherapy functions in a single unit, thereby improving overall convenience, versatility, and efficacy compared to conventional devices.

### SUMMARY OF THE INVENTION

According to a first aspect of the present invention, an applicator head for applying a liquid to a user's skin is provided. The applicator head comprising: a base having a connecting hole configured to connect to a liquid supply container; a main body rotatably connected to the base, an application surface at one end, and a liquid supply channel extending from the application surface to the other end, the liquid supply channel being rotatable to align or misalign with the connecting hole of the base to control liquid flow.

In one embodiment of the invention, the applicator head further comprising a roller having an elongated shape, a portion of which is located within a mounting groove located on the mounting groove and rotatably connected to the main body, a rotation axis of the roller extending along length of the main body.

In one embodiment of the invention, the main body comprises an outer shell and a support shell, the outer shell being hollow with a notch forming the mounting groove, and the support shell forming the mounting groove together with the outer shell.

In one embodiment of the invention, the support shell has an annular groove with a sealing ring, the support shell being sealed to the inner wall of the outer shell via the sealing ring.

In one embodiment of the invention, the applicator head further comprising a light therapy lamp with a plurality of light-emitting elements, each arranged within a space defined by surrounding light-shielding walls of the support shell.

According to a second aspect of the present invention, an applicator head for applying a liquid to a user's skin is provided. The applicator head comprising: a base having a connecting hole configured to connect to a liquid supply container; a main body rotatably connected to the base, a mounting groove at one end, and a liquid supply channel extending from the mounting groove to the other end, the liquid supply channel being rotatable to align or misalign with the connecting hole of the base to control liquid flow; and a roller having an elongated shape, a portion of which is located within the mounting groove and rotatably connected to the main body, the rotation axis of the roller extending along the length of the main body.

In one embodiment of the invention, the main body comprises an outer shell and a support shell, the outer shell being hollow with a notch forming the mounting groove, and the support shell forming the mounting groove together with the outer shell.

In one embodiment of the invention, the support shell has an annular groove with a sealing ring, the support shell being sealed to the inner wall of the outer shell via the sealing ring.

In one embodiment of the invention, the applicator head further comprising a light therapy lamp with a plurality of light-emitting elements, each arranged within a space defined by surrounding light-shielding walls of the support shell.

In one embodiment of the invention, the main body includes a mounting bracket, a circuit board, and a battery, the mounting bracket comprising a first mounting portion holding the circuit board and battery, and a second mounting portion engaging with a connecting sleeve of the base to connect the liquid supply container.

In one embodiment of the invention, the main body is rotatably connected to the base by engagement of a first stepped surface provided on an inner wall of a second mounting portion of the main body and a second stepped surface provided on a connecting sleeve of the base, the stepped surfaces cooperating to provide axial retention while permitting relative rotation of the main body with respect to the base.

In one embodiment of the invention, the support shell has a liquid supply tube forming the liquid supply channel, the end of the tube being plugged into a socket of the first mounting portion and sealingly abutting a sealing gasket.

In one embodiment of the invention, the liquid supply tube is eccentrically disposed relative to the rotational axis of the main body and the base, with the tube outlet positioned for maximum coverage along the mounting groove.

In one embodiment of the invention, the applicator head further comprising at least one stimulation element integrated with the roller or main body for mechanical, vibrational, or thermal stimulation.

In one embodiment of the invention, the stimulation element is controllable via a user interface including buttons, touch input, or a wireless/mobile application.

In one embodiment of the invention, the roller is interchangeable with rollers of varying texture or stiffness for different skin types or applications.

In one embodiment of the invention, the applicator head further comprising a micro-pump or electronic valve for precise regulation of liquid flow from the liquid supply container to the roller.

In one embodiment of the invention, the applicator head further comprising multiple liquid chambers in the liquid supply container, and the main body is configured to mix or selectively dispense liquids from different chambers.

In one embodiment of the invention, the applicator head further comprising a self-cleaning mechanism for the roller, including flushing channels or UV sterilization.

According to a third aspect of the present invention, a method for applying liquid to a user's skin is provided. The method comprising: providng an applicator device with an applicator head comprising a main body, a base, a liquid supply channel, roller and at least one stimulation element; rotating the main body relative to the base to connect the liquid supply channel to the liquid supply container; dispensing the liquid through the liquid supply channel to the roller; contacting the roller with the skin to apply the liquid; and activating a stimulation element integrated with the applicator head to provide mechanical, vibrational, or thermal stimulation to the skin.

In one embodiment of the invention, the method further comprising adjusting the intensity of the stimulation element or phototherapy lamp based on skin type, user preference, or pre-programmed routine.

In one embodiment of the invention, the method further comprising controlling the device remotely via a mobile application to adjust liquid dosage, roller speed, stimulation intensity, or phototherapy settings.

In the context of the specification, the term “comprising” (and grammatical variations thereof) is intended to be open-ended, meaning that the listed elements or steps are not exhaustive and do not exclude the presence of additional, unrecited elements or steps.

In the context of the specification, the terms “a,” “an,” and “the” are intended to cover both the singular and plural forms, unless otherwise indicated by context.

In the context of the specification, the term “or” is used inclusively, unless stated otherwise, meaning “and/or.”

In the context of the specification, the terms such as “axial,” “circumferential,” “radial,” “inner,” “outer,” “upper,” “lower,” “front,” “rear,” and the like are used for convenience to describe relative orientation and do not impose any absolute limitations on device orientation during use.

In the context of the specification, the term “applicator head” refers broadly to the structural portion of the device that interfaces with the user's skin and includes, but is not limited to, the base, main body, roller, liquid supply channel, stimulation elements, and/or phototherapy modules.

In the context of the specification, the term “roller” refers to any elongated or rotatable element adapted to transfer liquid to the skin and may include smooth rollers, textured rollers, interchangeable rollers, or rollers incorporating vibration, thermal, or magnetotherapy functions.

In the context of the specification, the term “liquid” is intended to encompass skincare products (e.g., creams, lotions, essences, serums), cleansing solutions (e.g., facial cleansers), therapeutic liquids (e.g., medicated formulations), or any other flowable substance suitable for topical application.

In the context of the specification, the term “liquid supply channel” includes any passage, conduit, tube, or equivalent structure that provides fluid communication between the liquid supply container and the roller or application surface, whether operated mechanically, electronically, or in combination.

In the context of the specification, the term “stimulation element” encompasses any module or structure configured to provide mechanical, vibrational, ultrasonic, microcurrent, piezoelectric, thermal, or magnetotherapeutic stimulation, either individually or in combination.

In the context of the specification, the term “phototherapy lamp” or “light therapy module” refers to any arrangement of light-emitting elements, including LEDs, OLEDs, or other light sources, capable of delivering therapeutic light wavelengths such as red, blue, green, or near-infrared, either continuously or in a programmable pattern.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others. The LEDs may also include UV-emitting diodes specifically selected for antimicrobial action in disinfection modules integrated into the multifunctional physiotherapy comb device.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminium, Boron, Zinc Selenide, etc., in pure form or doped with elements such as Aluminium and Indium, etc. For example, red and amber colours are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider colour gamut. White and other colored lightings may also be produced using phosphor coatings such as Yttrium Aluminium Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of the specification, the term “self-cleaning mechanism” refers to any arrangement for cleaning or sterilizing the applicator head or roller, including but not limited to flushing channels, liquid rinsing, and UV or germicidal irradiation.

In the context of the specification, the term “connected,” “coupled,” “engaged,” “mounted,” or similar language is intended to include direct and indirect connections, couplings, or engagements, whether permanent or detachable, unless expressly limited otherwise.

In the context of the specification, the terms “user interface” and “control module” include any physical button, switch, touch-sensitive surface, display, or wireless communication module (e.g., Bluetooth®, Wi-Fi®) that enables user input, remote control, or feedback regarding device operation.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user.

### BRIEF DESCRIPTION OF THE ACCOMPANYING DRAWINGS

In order to more clearly illustrate the technical solutions in the embodiments of the present invention, the drawings that need to be used in the embodiments or prior art descriptions will be briefly introduced below, and it is obvious that the drawings in the following descriptions are only some embodiments of the present invention, and for those skilled in the art, other drawings can also be obtained according to these drawings without paying creative labor.

FIG. 1 is a schematic perspective view of an applicator device according to an embodiment of the present invention.

FIG. 2 is a schematic perspective view of the applicator head of an applicator device shown in FIG. 1.

FIG. 3 is a cross-sectional view of the applicator head shown in FIG. 2.

FIG. 4 is an enlarged view of portion A in FIG. 3, showing the detailed structure of the mounting groove and roller assembly.

FIG. 5 is an exploded perspective view of the applicator head shown in FIG. 2, illustrating the arrangement of the shell, supporting shell, mounting bracket, and phototherapy lamp.

FIG. 6 is another cross-sectional schematic of the applicator head of FIG. 2, with the phototherapy lamp removed for clarity.

FIG. 7 is a schematic view showing the structure of the supporting shell of the applicator head as shown in FIG. 5.

FIG. 8 is a schematic perspective view of the mounting bracket of the applicator head as shown in FIG. 5.

FIG. 9 is a schematic view showing a cut-out portion of the mounting bracket illustrated in FIG. 8.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure is practiced. These embodiments are described in detail to enable those skilled in the art to practice the invention illustrated in the disclosure. It is to be understood that other embodiments are utilized, and other changes are made, without departing from the spirit or scope of the present disclosure. The following detailed description is therefore not to be taken in a limiting sense, and the scope of the present invention disclosure is defined by the appended claims. Embodiments of the claims, however, may be embodied in many different forms and should not be construed as limited to the embodiments set forth herein.

It should be noted that when an element is referred to as being “fixed to” or “disposed on” another element, it is directly or indirectly attached to the other element. When an element is referred to as being “connected to” another element, it is directly or indirectly connected to the other element.

Embodiments of the present invention disclose an applicator head and an applicator device for controlled and precise application of liquids onto the skin, including but not limited to skincare products such as eye cream, face cream, essence, and cleansing liquids such as facial cleansers. The invention integrates ergonomic design, liquid delivery, and multi-modal stimulation elements to provide enhanced skin treatment. The applicator head comprises a base configured for connection with a liquid supply container and a flattened main body designed for comfortable handling. The main body includes an elongated roller rotatably mounted along its length, which facilitates smooth and uniform application of the liquid onto the skin. The roller, in combination with a rotatable main body and eccentrically arranged liquid supply channel, allows selective control of liquid flow, preventing unintended leakage during transport and storage.

In an embodiment, the liquid supply channel is rotatably connected with the base such that the channel can align with or be staggered from the connecting port of the liquid container. In the aligned position, liquid flows through the supply channel to the roller for application, whereas in the staggered position, flow is blocked, preventing accidental spillage. This arrangement can be further enhanced with electronic valves or micro-pumps for automated and precise liquid dispensing, as well as flow sensors to monitor and provide feedback on the amount of liquid applied. Multi-chamber liquid containers can also be incorporated to allow mixing or sequential application of multiple liquids, enhancing the versatility of skincare or therapeutic routines.

In an embodiment, the applicator head comprises one or more stimulation elements. The stimulation elements are selected from a group consisting of, but not limited to, (i) mechanical stimulation through rollers with variable stiffness or interchangeable textures to provide massage, exfoliation, or gentle liquid application; (ii) vibrational or ultrasonic modules to enhance absorption and tactile feedback; and (iii) thermal elements for localized heat therapy.

In an embodiment, the applicator head comprises phototherapy modules with multiple light wavelengths, such as red, blue, and near-infrared LEDs. These elements can operate independently or synergistically to enhance therapeutic outcomes, improve circulation, promote skin rejuvenation, and complement the applied liquids.

The applicator device houses a mounting bracket, circuit board, and battery to control the operation of the roller, stimulation elements, phototherapy modules, and automated liquid dispensing. User interfaces such as buttons or touch controls enable the selective activation of the various elements and safety features. Safety improvements include roller lift detection to stop liquid flow when the applicator device is not in contact with the skin, an anti-slip casing for secure handling, and a lid that prevents exposure of the roller when not in use. Wireless connectivity via a mobile application can also be provided to track usage, remotely control stimulation and phototherapy settings, and recommend personalized routines based on skin type or condition.

In an embodiment, for maintaining hygiene, self-cleaning mechanisms are provided in the applicator head. The self-cleaning mechanisms comprise flushable internal channels or UV sterilization of the roller, ensuring the applicator device remains clean and suitable for repeated use. The applicator head is designed for modular assembly, enabling easy replacement of rollers, stimulation modules, or phototherapy units. The combination of a flattened ergonomic main body, elongated roller, rotatable base, and multi-modal stimulation elements provides a versatile, portable, and effective solution for skincare, cosmetic, or therapeutic treatments.

Applications of the invention include facial and body skincare, massage, phototherapy, thermal therapy, and multi-step liquid treatment routines. The invention provides a controlled, precise, and hygienic means for delivering liquids to the skin while simultaneously offering mechanical, thermal, and phototherapeutic stimulation. By integrating smart liquid control, adjustable stimulation elements, phototherapy, ergonomic handling, mobile connectivity, and self-cleaning features, the invention delivers enhanced skin treatment efficacy, safety, and user convenience.

Several embodiments of the present invention will now be described in detail with references to FIGS.

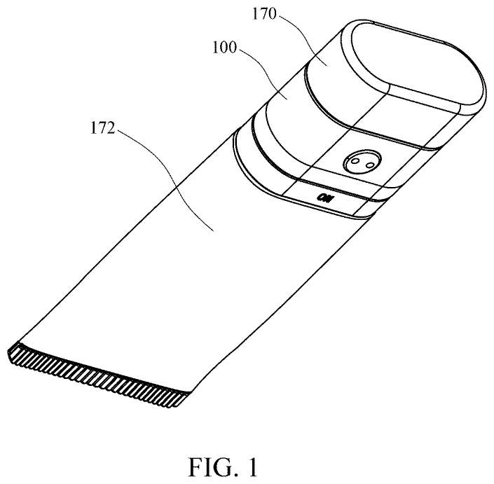

FIG. 1 illustrates an embodiment of the present invention, showing an applicator device with an applicator head 100. The applicator head 100 includes a cover 170 and a liquid supply container 172. The applicator head 100 is configured to apply liquid from a liquid supply container 172 onto the surface of a user's skin. The cover 170 protects the application head 100 from dust and dirt, which can lead to contamination when the applicator head 100 is not in use. The liquid may include, but is not limited to, skincare products such as eye cream, face cream, essence, or other topical formulations. Additionally, the liquid may comprise cleansing solutions, including facial cleansers, thereby allowing the device to serve both cosmetic and skincare purposes.

Referring to FIG. 1 and FIGS. 2 to 4, in an embodiment, the applicator device comprises the applicator head 100 in combination with a liquid supply container 172. The base 102 of the applicator head 100 is coupled to the liquid supply container 172. The liquid supply container 172 includes a liquid outlet provided with external threads on its periphery. The connecting sleeve 106 of the base 102 is correspondingly formed with internal threads, such that the liquid outlet of the liquid supply container 172 is threadably engaged with the connecting sleeve 106, establishing fluid communication between the liquid supply container 172 and the connecting hole 104. Additionally, in an embodiment, the liquid outlet of the liquid supply container 172 may be configured for direct insertion into the connecting sleeve 106 to form a frictional or interference fit, thereby providing a sealed connection without requiring threaded engagement.

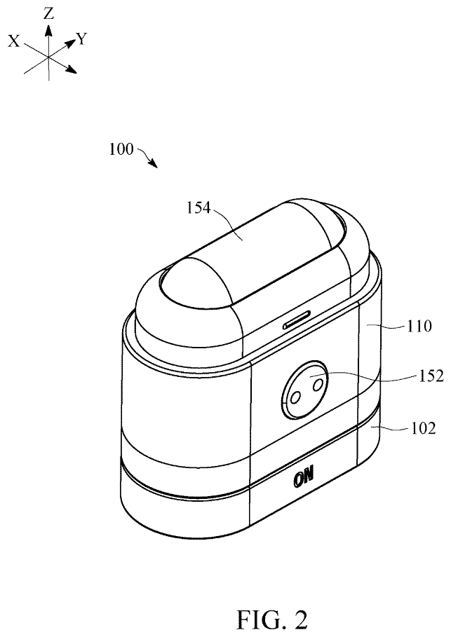

The applicator head 100 comprises a base 102, a main body 110, and a roller 154. The base 102 is formed with a connecting hole 104, which is configured to couple with the liquid supply container 172 such that the connecting hole 104 is in fluid communication with the interior of the container. The main body 110 is generally flat in form and includes a mounting groove 112 disposed at one end thereof in the height direction (Z-direction in FIG. 2). The opposite end of the main body 110 is rotatably connected to the base 102. A liquid supply channel 114 is formed within the main body 110, one end of which extends through a wall portion of the mounting groove 112, while the other end is selectively alignable with the connecting hole 104 of the base 102 as the main body 110 rotates relative to the base 102. The roller 154 is elongated and oriented along the length of the main body 110 (X-direction in FIG. 2). A portion of the roller 154 is received within the mounting groove 112 and is rotatably supported therein, such that its rotational axis extends along the lengthwise direction of the main body 110. In the illustrated embodiment, the main body 110 exhibits a flat configuration with a lengthwise direction (X-direction) greater than its widthwise direction (Y-direction), thereby providing a thin, planar morphology.

The applicator head 100 is distinguished by the provision of the large, elongated roller 154, the rotation axis of which extends along its length. This structural arrangement affords a reduced rotation radius, thereby enabling a smooth, rapid, and flexible rolling motion conducive to improved massaging comfort for the user. The ease of rotation also facilitates the transfer of liquid from the mounting groove 112 onto the user's skin. Moreover, the flat profile of the main body 110 is morphologically compatible with the elongated roller 154, permitting the roller 154 to be efficiently positioned to maximize space utilization and ergonomic benefit. The flat configuration of the main body 110 further enables the user to grip it conveniently along its width, thereby enhancing handling comfort and reducing operational effort.

In an embodiment, the roller of the applicator head 100 is designed to be interchangeable with alternative rollers having varying textures, surface contours, or stiffness levels. For example, a soft silicone roller may be employed for sensitive skin, while a firmer roller may be used for deeper massage effects. Textured rollers may provide enhanced exfoliation or improved liquid spreading. The rollers may be removably mounted within the mounting groove through snap-fit, magnetic coupling, or threaded engagement, allowing convenient replacement by the user. This interchangeability ensures the adaptability of the applicator head to a wide range of skin types and treatment needs.

The rotatable coupling between the main body 110 and the base 102 enables selective fluid communication between the liquid supply channel 114 and the connecting hole 104. In a connected position, the liquid supply channel 114 and the connecting hole 104 are brought into maximum overlap, thereby establishing a passage through which the liquid contained in the liquid supply container 172 flows sequentially through the connecting hole 104, the liquid supply channel 114, and into the mounting groove 112, from which it is transferred by the roller 154 to the skin surface. In an offset position, the liquid supply channel 114 is displaced from the connecting hole 104 to a non-overlapping state, thereby blocking the outlet of the container. In this condition, even if external pressure is applied to the liquid supply container 172, liquid discharge is prevented. Consequently, by simple rotation of the main body 110, the liquid supply container 172 can be effectively closed when not in use, ensuring reliable sealing, leak prevention, and convenience for portable applications.

Additionally, the liquid supply container 172 may include multiple chambers, each containing a different liquid formulation such as serum, lotion, or medicinal solution. The main body of the applicator head 100 may include a valve system or mixing channel to selectively dispense liquid from a single chamber or to combine liquids from multiple chambers before delivery to the roller 154. The user may choose the active chamber through the user interface or a mechanical selector switch. This arrangement enables flexible treatment regimens, including sequential application of multiple liquids or blending formulations to achieve synergistic therapeutic effects.

In the illustrated embodiment, the liquid supply channel 114 and the connecting hole 104 are arranged in an eccentric position relative to the rotational axis defined between the base 102 and the main body 110. This eccentric configuration permits the liquid supply channel 114 and the connecting hole 104 to be selectively brought into alignment or displaced into a fully offset position through relative rotation. Between these two positions, an intermediate state may be established wherein the liquid supply channel 114 and the connecting hole 104 are only partially overlapped, thereby providing a variable connection area ranging continuously from a maximum overlap to zero overlap.

In certain embodiments, the rotational axis between the base 102 and the main body 110 is oriented along the height direction of the main body 110. In such cases, the relative rotation angle required to transition from the fully connected position to the fully offset position may be approximately 180°. Alternatively, in other embodiments, the rotation angle may be 90° or another predetermined value depending on the structural design of the applicator head 100.

In an embodiment, the applicator head 100 may incorporate a micro-pump or electronically actuated valve positioned within the liquid supply channel 114 or adjacent to the liquid outlet of the liquid supply container 172. The micro-pump or valve may be controlled by the circuit board 150 in response to user input from the interface, thereby regulating the amount and timing of liquid delivered to the roller 154. Such precision control prevents excess liquid dispensing, reduces waste, and allows accurate dosing of therapeutic or cosmetic formulations. The pump or valve may also be synchronized with the operation of the stimulation element to coordinate liquid application with specific treatment modes.

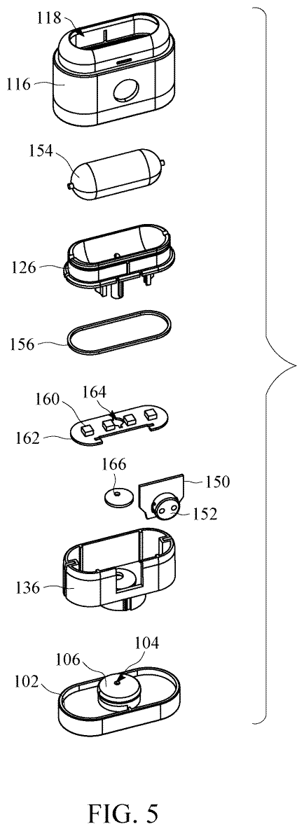

Referring to FIG. 5, in an embodiment, the main body 110 comprises an outer shell 116 and a support shell 126. The outer shell 116 is formed as a hollow structure, with one end being open to define a notch 118 that accommodates the mounting groove 112. The support shell 126 is disposed within the hollow interior of the outer shell 116 and, in cooperation with the outer shell 116, defines the mounting groove 112 for receiving and rotatably supporting the roller 154.

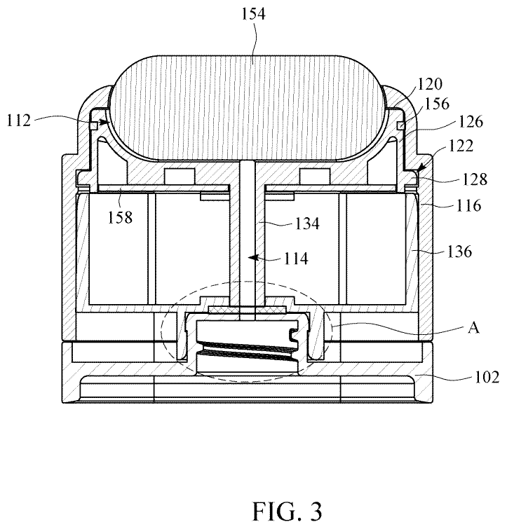

Referring to FIG. 3, a retaining flange 120 is disposed on the inner wall of the outer shell 116 adjacent to the notch 118. The support shell 126 abuts the surface of the retaining flange 120 facing away from the notch 118, thereby preventing separation of the support shell 126 from the outer shell 116 along the direction of the notch 118. In certain embodiments, the inner wall of the outer shell 116 may be stepped to define multiple retaining flanges 120, thereby forming a multi-position retaining mechanism. Additionally, a retaining rib 128 is formed at the end of the support shell 126 opposite the notch 118, while a corresponding retaining groove 122 is formed on the inner wall of the outer shell 116. During assembly, the support shell 126 may be inserted upward into the outer shell 116 until the support shell 126 abuts against the retaining flange 120 and the retaining rib 128 is engaged with the retaining groove 122, thereby restricting vertical displacement of the support shell 126. In alternative embodiments, the support shell 126 and the outer shell 116 may be secured by snap-fit engagement, threaded fasteners, or equivalent fixing means.

Referring to FIGS. 3 and 7, the support shell 126 further defines an annular groove 130 surrounding the notch 118, within which a sealing ring 156 is seated. The sealing ring 156 establishes a fluid-tight engagement between the support shell 126 and the inner wall of the outer shell 116, thereby preventing ingress of water, dust, or other contaminants into the interior of the outer shell 116 through the notch 118 of the mounting groove 112. The annular groove 130 also prevents vertical displacement of the sealing ring 156, ensuring long-term stability and reliability of the seal.

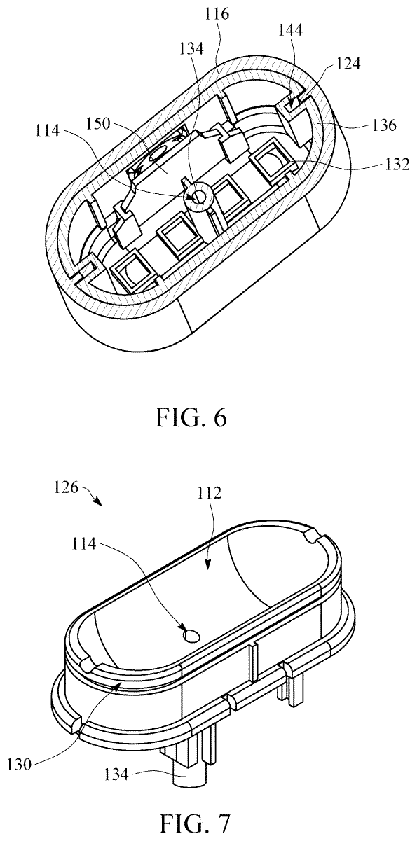

Referring to FIGS. 5 and 6, in an embodiment, the support shell 126 is fabricated from a light-transmissive material. A plurality of surrounding walls 132 is provided on the side of the support shell 126 opposite the notch 118, the walls being spaced apart along the lengthwise direction of the main body 110. The applicator head 100 further includes a phototherapy lamp 158 comprising a plurality of light-emitting elements 160 arranged in one-to-one correspondence with the surrounding walls 132. Each light-emitting element 160 is positioned within a space defined by the surrounding walls 132. The surrounding walls 132 may be inherently light-shielding structures, or alternatively, a light-shielding member, such as light-shielding cotton or a light-shielding coating, may be disposed on the inner surface of the surrounding walls 132. The shielding structure or member confines light emitted by the light-emitting elements 160, thereby concentrating and directing the light outwardly through the support shell 126 to achieve effective therapeutic illumination.

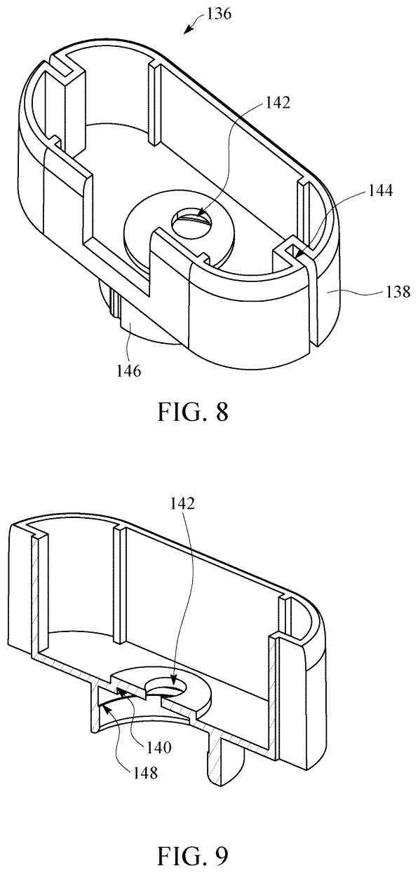

Referring to FIGS. 5 and 8, in an embodiment, the main body 110 further comprises a mounting bracket 136, a circuit board 150, and a power supply such as a battery (not shown). The mounting bracket 136 is disposed within the outer shell 116 between the support shell 126 and the base 102, and includes a first mounting portion 138 and a second mounting portion 146. The first mounting portion 138 is a cavity structure open toward the support shell 126, configured to accommodate and secure the circuit board 150 and the battery. In certain implementations, the first mounting portion 138 may define a slot for insertion of the circuit board 150 and the battery. Alternatively, a latch mechanism may be employed for retention, or the battery may be affixed to the first mounting portion 138 using an adhesive.

Referring further to FIG. 6, the first mounting portion 138 contacts the inner wall of the outer shell 116 and is provided with a guide groove 144. Corresponding guide ribs 124 are formed on the inner wall of the outer shell 116, and are configured for insertion into the guide groove 144. Both the guide groove 144 and the guide ribs 124 extend along the height direction of the main body 110, thereby facilitating precise assembly. The side of the outer shell 116 opposite the mounting groove 112 is open to allow insertion and installation of the bracket assembly.

During assembly, the mounting bracket 136 is introduced into the outer shell 116 through the open side of the shell, with the guide ribs 124 sliding into the corresponding guide grooves 144 to ensure precise positioning. The main body 110 further includes a button 152 electrically coupled to the circuit board 150, the button 152 serving as a control element for switching the phototherapy lamp 158 on and off.

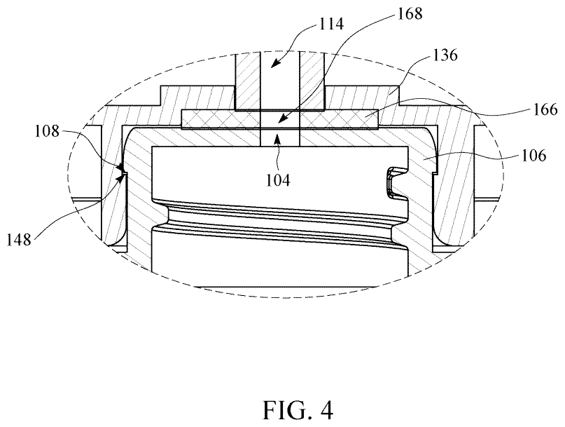

Referring to FIGS. 4 and 9, the second mounting portion 146 is disposed on the side of the first mounting portion 138 facing the base 102. The second mounting portion 146 is annular in configuration, and an inwardly facing first stepped surface 148 is formed on its inner wall, with the first stepped surface 148 oriented away from the base 102. The base 102 is provided with a connecting sleeve 106 dimensioned to be received within the second mounting portion 146. The connecting sleeve 106 includes a second stepped surface 108, which abuts the first stepped surface 148 when the connecting sleeve 106 is fully inserted into the second mounting portion 146. This abutting engagement prevents axial separation of the connecting sleeve 106 from the second mounting portion 146, thereby securing the base 102 to the main body 110. Such a stepped-surface retention structure facilitates both reliable alignment of the base 102 and the main body 110 during installation and convenient disassembly when required. This stepped-surface engagement forms a stable rotary joint that secures the base 102 and main body 110 together while simultaneously permitting relative rotation about the central axis of the connecting sleeve 106.

The stepped engagement provides both axial retention and rotary support, ensuring that the main body 110 remains assembled with the base 102 without requiring additional fasteners, while still allowing smooth rotation to selectively align or offset the liquid supply channel 114 with the connecting hole 104. The cooperation of the first stepped surface 148 and the second stepped surface 108 thus serves a dual function of structural retention and controlled rotation, enabling convenient switching between open and closed fluid states.

The first mounting portion 138 further defines a receiving groove 140 on the side facing the base 102, with the second mounting portion 146 surrounding the receiving groove 140. A sealing gasket 166 is disposed within the receiving groove 140. The support shell 126 is provided with a liquid supply tube 134, which forms part of the liquid supply channel 114. The first mounting portion 138 includes a socket 142 extending through the bottom wall of the receiving groove 140, into which the end of the liquid supply tube 134 is inserted and sealed against the sealing gasket 166. The sealing gasket 166 is provided with a communication hole 168 corresponding to the socket 142, thereby maintaining fluid communication while ensuring sealing integrity. The terminal end of the connecting sleeve 106 also abuts and seals against the sealing gasket 166. In this configuration, the sealing gasket 166 is retained between the first mounting portion 138 and the connecting sleeve 106, and further positioned by the walls of the receiving groove 140, which limit any radial displacement. This arrangement ensures stable sealing performance while preventing leakage during operation.

In an embodiment, the connecting sleeve 106 may be closed at one end and provided with a connecting hole 104, which is configured to align with and communicate through the corresponding communication hole 168 formed in the sealing gasket 166. Alternatively, the connecting sleeve 106 may be formed as a through-sleeve with openings at both ends. The provision of the sealing gasket 166 ensures a reliable and leak-proof connection between the connecting hole 104 of the connecting sleeve 106 and the liquid supply channel 114, thereby preventing unintended leakage during operation.

In an embodiment, the liquid supply tube 134 is disposed eccentrically with respect to the rotational axis defined between the main body 110 and the base 102. The end of the liquid supply tube 134, which extends through the wall of the mounting groove 112, is positioned substantially at the longitudinal midpoint of the mounting groove 112, while being offset from the central widthwise axis of the mounting groove 112. By positioning the outlet of the liquid supply tube 134 at the longitudinal midpoint, the outlet-to-end distance in both lengthwise directions is equal and minimized, thereby allowing the discharged liquid to flow toward both ends of the mounting groove 112 and ensuring wide coverage. By offsetting the outlet relative to the widthwise midpoint, the effective width dimension of the structure is reduced, while still permitting the liquid to adequately reach both widthwise sides of the groove.

In an embodiment, the applicator head 100 is further provided with a self-cleaning mechanism for the roller 154. The self-cleaning mechanism may include flushing channels integrated into the mounting groove, allowing a cleaning solution or water to flow through and wash away residual liquid from the roller surface. Additionally, a UV sterilization module may be positioned adjacent to the roller or within the support shell to irradiate the roller with germicidal ultraviolet light, thereby reducing microbial contamination. The combination of flushing and UV sterilization ensures hygienic reuse of the applicator head and extends its operational life without manual disassembly.

Furthermore, the phototherapy lamp 158 comprises a lamp board 162, on which the light-emitting elements 160 are mounted. The support shell 126 is provided with a clip configured to engage the side of the lamp board 162 opposite the notch 118. The open end of the first mounting portion 138 also abuts the side of the lamp board 162 opposite the notch 118. In this configuration, the clip and the first mounting portion 138 jointly support the lamp board 162, with the clip further serving to secure the lamp board 162 in position and prevent undesired movement. Referring to FIGS. 3 and 5, the lamp board 162 is further provided with a sleeve hole 164 adapted to receive insertion of the liquid supply tube 134. To prevent relative rotation, anti-rotation ribs may be formed on the outer surface of the liquid supply tube 134, while complementary anti-rotation grooves may be provided on the inner wall of the sleeve hole 164, the engagement of which prevents rotational displacement of the liquid supply tube 134 relative to the lamp board 162.

In an embodiment, the applicator head 100 may further be provided with one or more stimulation elements configured to enhance the therapeutic effect of the applied liquid formulations. The stimulation elements are configured to deliver one or more therapeutic modalities, including vibration stimulation, microcurrent stimulation, ultrasonic wave stimulation, phototherapy, piezoelectric therapy, and magnetotherapy. The stimulation elements may be disposed within the main body 110, the roller 154, or in association with the support shell 126, and are operatively coupled to the circuit board 150 for power and control. Such integration allows simultaneous or selective activation of stimulation functions while liquid is being applied to the skin. The stimulation elements can operate independently or in combination, depending on user settings or treatment requirements.

The stimulation elements may include vibrational modules configured to generate oscillatory motion of the roller 154 or the main body 110. Vibrational stimulation improves skin microcirculation, promotes relaxation of facial muscles, and facilitates absorption of the applied liquid. In another embodiment, the stimulation element comprises a microcurrent generator electrically connected to skin-contact electrodes, delivering low-level electrical currents to stimulate cellular activity, enhance collagen production, and support skin rejuvenation.

In an embodiment, ultrasonic transducers are integrated into the applicator head 100 to generate high-frequency ultrasonic waves. These ultrasonic vibrations induce cavitation effects and micro-massage within the skin, thereby increasing the permeability of the skin barrier and allowing deeper penetration of active ingredients. Similarly, piezoelectric modules may be employed, wherein piezoelectric ceramics convert electrical energy into mechanical oscillations or ultrasonic waves, further enhancing therapeutic efficiency while maintaining compact structural design.

Additionally, phototherapy lamp 158 may be supplemented with magnetotherapy units, wherein one or more permanent magnets or electromagnets are incorporated within the outer shell 116 or roller 154. Magnetotherapy provides magnetic field stimulation that may improve circulation, reduce inflammation, and support skin healing processes. When combined with vibration, microcurrent, ultrasonic, or light therapy, magnetotherapy creates a multi-modal treatment environment that delivers synergistic effects. The integration of these diverse stimulation elements with the liquid delivery mechanism provides users with a comprehensive skincare and therapeutic device capable of addressing cosmetic, dermatological, and wellness needs in both consumer and clinical applications.

Additionally, the stimulation element is controllable via a user interface. The user interface may include physical buttons disposed on the outer shell of the main body, capacitive or resistive touch input regions, or wireless control through a mobile application communicating with the circuit board via Bluetooth® or Wi-Fi®. This arrangement enables the user to selectively activate, deactivate, or adjust the operational parameters of the stimulation element, such as vibration intensity, microcurrent amplitude, ultrasonic frequency, or light therapy wavelength. The inclusion of such control interfaces allows for customized treatment settings suited to different therapeutic or cosmetic applications.

The present invention, therefore, provides a multifunctional applicator head and applicator device capable of delivering controlled liquid application in combination with mechanical stimulation and phototherapy. By integrating ergonomic handling, leak-proof sealing structures, eccentric liquid distribution, and modular stimulation components, the device ensures precision, hygiene, and versatility. The applicator device has a broad industrial applicability in the fields of cosmetics, dermatology, personal care, and therapeutic treatment devices. It can be manufactured at scale using conventional molding, sealing, and electronic assembly processes, making it suitable for both consumer and clinical markets. The applicator device enhances the effectiveness of skincare formulations, supports multifunctional treatment regimens, and improves user convenience. As such, the invention addresses long-standing challenges in liquid applicators by providing a reliable, portable, and technologically advanced solution, thereby offering significant commercial and practical value in the global health and beauty industry.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. An applicator head for applying a liquid to a user's skin, comprising:
a base having a connecting hole configured to connect to a liquid supply container;
a main body rotatably connected to the base, an application surface at one end, and a liquid supply channel extending from the application surface to the other end, the liquid supply channel being rotatable to align or misalign with the connecting hole of the base to control liquid flow.

2. The applicator head of claim 1, further comprising a roller having an elongated shape, a portion of which is located within a mounting groove located on the mounting groove and rotatably connected to the main body, a rotation axis of the roller extending along length of the main body.

3. The applicator head of claim 2, wherein the main body comprises an outer shell and a support shell, the outer shell being hollow with a notch forming the mounting groove, and the support shell forming the mounting groove together with the outer shell.

4. The applicator head of claim 3, wherein the support shell has an annular groove with a sealing ring, the support shell being sealed to an inner wall of the outer shell via the sealing ring.

5. The applicator head of claim 4, further comprising a light therapy lamp with a plurality of light-emitting elements, each arranged within a space defined by surrounding light-shielding walls of the support shell.

6. An applicator head for applying a liquid to a user's skin, comprising:
a base having a connecting hole configured to connect to a liquid supply container;
a main body rotatably connected to the base, a mounting groove at one end, and a liquid supply channel extending from the mounting groove to the other end, the liquid supply channel being rotatable to align or misalign with the connecting hole of the base to control liquid flow; and
a roller having an elongated shape, a portion of which is located within the mounting groove and rotatably connected to the main body, a rotation axis of the roller extending along length of the main body.

7. The applicator head of claim 6, wherein the main body comprises an outer shell and a support shell, the outer shell being hollow with a notch forming the mounting groove, and the support shell forming the mounting groove together with the outer shell.

8. The applicator head of claim 7, wherein the support shell has an annular groove with a sealing ring, the support shell being sealed to an inner wall of the outer shell via the sealing ring.

9. The applicator head of claim 8, further comprising a light therapy lamp with a plurality of light-emitting elements, each arranged within a space defined by surrounding light-shielding walls of the support shell.

10. The applicator head of claim 9, wherein the main body includes a mounting bracket, a circuit board, and a battery, the mounting bracket comprising a first mounting portion holding the circuit board and battery, and a second mounting portion engaging with a connecting sleeve of the base to connect the liquid supply container.

11. The applicator head of claim 10, wherein the main body is rotatably connected to the base by engagement of a first stepped surface provided on an inner wall of a second mounting portion of the main body and a second stepped surface provided on a connecting sleeve of the base, the stepped surfaces cooperating to provide axial retention while permitting relative rotation of the main body with respect to the base.

12. The applicator head of claim 10, wherein the support shell has a liquid supply tube forming the liquid supply channel, the end of the tube being plugged into a socket of the first mounting portion and sealingly abutting a sealing gasket.

13. The applicator head of claim 12, wherein the liquid supply tube is eccentrically disposed relative to the rotational axis of the main body and the base, with a tube outlet positioned for maximum coverage along the mounting groove.

14. The applicator head of claim 6, further comprising at least one stimulation element integrated with the roller or main body for mechanical, vibrational, or thermal stimulation.

15. The applicator head of claim 14, wherein the stimulation element is controllable via a user interface including buttons, touch input, or a wireless/mobile application.

16. The applicator head of claim 6, further comprising a micro-pump or electronic valve for precise regulation of liquid flow from the liquid supply container to the roller.

17. The applicator head of claim 6, further comprising a self-cleaning mechanism for the roller, including flushing channels or UV sterilization.

18. A method for applying liquid to a user's skin, comprising:
providing an applicator device with an applicator head comprising a main body, a base, a liquid supply channel, a roller, and at least one stimulation element;
rotating the main body relative to the base to connect the liquid supply channel to the liquid supply container;
dispensing the liquid through the liquid supply channel to the roller;
contacting the roller with the skin to apply the liquid; and
activating a stimulation element integrated with the applicator head to provide mechanical, vibrational, or thermal stimulation to the skin.

19. The method of claim 14, further comprising adjusting intensity of the stimulation element or phototherapy lamp based on skin type, user preference, or pre-programmed routine.

20. The method of claim 14, further comprising controlling the device remotely via a mobile application to adjust liquid dosage, roller speed, stimulation intensity, or phototherapy settings.
